(19) United States
(12) Patent Application Publication
(10) Pub. No.: US 2015/0118197 A1
(43) Pub. Date: Apr. 30, 2015

(54) SCAFFOLD

(71) Applicant: University of Sheffield, Sheffield, South Yorkshire (GB)

(72) Inventors: Frederik Claeyssens, Sheffield (GB); Ilida Ortega, Sheffield (GB); Sheila MacNeil, Sheffield (GB); Anthony Ryan, Sheffield (GB)

(21) Appl. No.: 14/398,324

(22) PCT Filed: May 1, 2013
(86) PCT No.: PCT/GB2013/051126

§ 371 Date: Oct. 31, 2014

(30) Foreign Application Priority Data
May 2, 2012 (GB) 1207723.6

(51) Int. Cl. A61K 9/00; C12N 5/079; A61K 35/30

(52) U.S. Cl. CPC A61K 9/0051; A61K 35/30; C12N 5/0621; C12N 2533/40
USPC 424/93.7; 435/396; 264/465; 264/401

(57) ABSTRACT

The invention provides a method for producing an electrospun scaffold, comprising electrospinning a polymer or co-polymer onto a template comprising a conductive collector having a three dimensional pattern thereon, wherein said electrospun polymer or co-polymer preferentially deposits onto said three dimensional pattern.

This invention relates to a method producing an electrospun scaffold. Also provided are scaffolds and uses thereof.

### BACKGROUND

Stem cells are thought to be located in defined microenvironments which are chemically, topologically and biologically well-characterized. These microenvironments are called niches (Fuchs, Tumbar et al. 2004). In the cornea, stem cells are situated in the limbus, specifically in microenvironments known as Palisades of Vogt (Ebato, Friend et al. 1987; Cotsarelis, Cheng et al. 1989).

Corneal disruption can occur for several reasons but in many cases the pool of limbal stem cells are lost, often due to damage to the epithelial niches or crypts. In the absence of a renewable population of these cells, cells from the conjunctiva migrate over the cornea producing essentially a scar tissue which is often heavily inflamed and is opaque and vascularised causing reduced vision or even blindness. Accordingly there has been considerable effort over the last 50 years in the development of approaches to assist in corneal regeneration.

The frontline treatment is that of corneal transplantation with a donor cornea. The initial success rate of transplantation with donor corneas can be very high (93%) throughout the first year post transplant but these tend to decrease to around 72% after 5 years. While the last 50 years of using this show it to be a highly successful transplant their success relies on these grafts being resurfaced with the patient's own limbal epithelial cells. Accordingly for patients who lack any residual LEC these will fail.

In some specialist centres the donor cornea is sometimes combined with cultured stem cell therapy. Essentially the corneal graft provides a restoration of a normal cornea wound bed and the cultured cell therapy then replaces the stem cell population.

For other patients delivery of LEC (usually on amniotic membrane used as a biological carrier) onto the denuded surface of the cornea will work. For culture of limbal stem cells these are usually cultivated from the limbus of the contralateral eye if the defect is only in one eye or from donor corneas if the defect affects both eyes. Occasionally autologous buccal mucosa cells are used as an alternative to donor corneal, cells avoiding the need for immunosuppression which is essential if one uses donor cells.

In restoring cultured limbal epithelial cells to the cornea the majority of studies have placed these cells on amniotic membrane to ensure the initial attachment and survival of these cells and the amniotic membrane with cells facing outermost is then grafted onto the cornea. Initial results with these grafts are very good (greater than 85% in year 1) but the success rate drops to close to 45% after 3 years of implantation.

Several alternative materials have been explored as either permanent implants to replace damaged corneas or as alternative cell delivery carriers to replace the amniotic membrane. For the latter both natural extracellular matrix components and synthetic polymeric substrates have been reported as possible stem cell carriers. For example collagen based substrates were used by Dravida and Gaddipati et al. 2008 and MacLaughlin et al used collagen-phosphorylcholine substrates as corneal implants for studies of nerve regeneration in guinea pigs.

Regarding the use of polymers as carriers, Sitalaksmi et al (Sitalakshmi, Sudha et al. 2008) studied the thermoresponsive properties of a polyethylene glycol-based matrix (Mebiol gel) in rabbit eyes. Deshpande et al. reported the potential of using poly(lactide-co-glycolide) electrospun scaffolds as a biodegradable synthetic alternative to the use of the amniotic membrane (Deshpande, McKean et al.). Rabbit corneal cells attached well to these electrospun scaffolds containing microfibers of ⅔ micron.

The use of electrospun scaffolds is an extensively explored technique in tissue engineering. Electrospinning is a versatile fabrication process that uses a high voltage in between a syringe and a deposition target (or collector) to draw thin fibers from the material dispensed by the syringe. The deposition of those fibers in a specifically located collector permits the generation of 3D fibrous scaffolds (Shin, Hohman et al. 2001).

Electrospun scaffolds mimic to a high degree three-dimensional extracellular matrices. The 3D network of electrospun fibres provides cells with mechanical support and attachment; also, the dimensions of the electrospun fibres (from nano to micro depending on the working conditions) are of the order of the protein assemblies found on 3D natural extracellular matrices. These electrospun scaffolds provide cell cultures with a porous environment with a high surface area per unit volume.

The choice of polymer and electrospinning conditions can be tuned to give a wide range of the desired properties and morphologies of 3D scaffolds. Going beyond this, several authors have described the use of patterned collectors for the study of alignment and morphology of electrospun fibres (Vaquette and Cooper-White; Matthews, Wnek et al. 2002; Zussman, Theron et al. 2003; Li, Ouyang et al. 2005; Zhang and Chang 2007), for example using either rotating (Matthews, Wnek et al. 2002 and Zussman, Theron et al. 2003) or static collectors for fiber deposition.

Other authors (Vaquette and Cooper-White) have used patterned collectors to enhance cell penetration into the scaffolds.

However, there remains a need for an improved implantable device for delivering stem cells for regeneration purposes.

### BRIEF SUMMARY OF THE DISCLOSURE

In accordance with the present inventions there is provided a method for producing an electrospun scaffold, comprising electrospinning a polymer or co-polymer onto a template comprising a conductive collector having a three dimensional pattern thereon, wherein said electrospun polymer or co-polymer preferentially deposits onto said three dimensional pattern.

Preferably, said three dimensional pattern is non-conductive.

Preferably, said three dimensional pattern is dimensioned to provide an electrospun scaffold having at least one cavity therein capable of acting as a stem cell niche.

Preferably, said polymer or co-polymer is biodegradable. More preferably, said biodegradable polymer or copolymer is biocompatible.

Preferably, said biodegradable polymer is a collagen, a poly alpha ester, a polyorthoester or a polyanhydride or a copolymer thereof. Still more preferably, said biodegradable polymer or copolymer is cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde or a copolymer thereof.

Preferably said polymer is polylactate acid, or is polyglycolic acid.

Preferably, said copolymer is a copolymer of polylactate acid and polyglycolic acid. More preferably said copolymer is a poly(D,L-lactic-co-glycolide). Alternatively, said poly(D,L-lactic-co-glycolide) has a ratio of 75:25 lactide to glycolide or said poly(D,L-lactic-co-glycolide) has a ratio of 50:50 lactide to glycolide.

Preferably, said conductive collector is an aluminium sheet.

Preferably said three dimensional pattern is formed on said carrier by microfabrication. Preferably, said microfabrication is microstereolithography.

Preferably, said three dimensional pattern is formed from polyethylene glycol.

In a further aspect there is provided an electrospun scaffold having at least one cavity therein capable of acting as a stem cell niche produced in accordance with a method of the invention.

In a further aspect there is provided an electrospun scaffold, comprising a biodegradable polymer or co-polymer, wherein said scaffold comprises at least one cavity therein capable of acting as a stem cell niche.

Preferably said biodegradable polymer or copolymer is biocompatible. Preferably said biodegradable polymer is a collagen, a poly alpha ester, a polyorthoester or a polyanhydride or a copolymer thereof. More preferably, said biodegradable polymer or copolymer is cellulose ether, cellulose, cellulosic ester, fluorinated polyethylene, phenolic, oly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde or a copolymer thereof.

Preferably said polymer is polylactide or polylactic acid, or polyglycolic acid.

Preferably said copolymer is a copolymer of polylactide or polylactic acid and polyglycolic acid. More preferably, said copolymer is a poly(D,L-lactic-co-glycolide). Preferably, said poly(D,L-lactic-co-glycolide) has a ratio of 75:25 lactide to glycolide.or said poly(D,L-lactic-co-glycolide) has a ratio of 50:50 lactide to glycolide.

Preferably said scaffold further comprises at least one stem cell. Preferably, said at least one stem cell is a limbal stem cell. Alternatively, said at least one stem cell is mesenchymal stem cell.

In a further aspect, there is provided an electrospun scaffold according to the invention, for use as a medicament.

In a further aspect, there is provided an electrospun scaffold in accordance with the invention, for use in corneal replacement.

In a further aspect, there is provided an electrospun scaffold in accordance with the invention, for use in the treatment of ocular injury.

In a further aspect, there is provided an electrospun scaffold in accordance with the invention, for use in the treatment of a wound. Preferably, said wound is a chronic wound or an acute wound.

### BRIEF DESCRIPTION OF THE DRAWINGS

Embodiments of the invention are further described hereinafter with reference to the accompanying drawings, in which:

FIG. 1 is a schematic representation of the effect produced on the deposition of the fibres by varying the height of the PEGDA collectors. The inner area of the scaffolds became thinner when the height of the collector increased, the thickness of the electrospun outer ring being constant. Figures A, B, C and D correspond to substrates of height 0.9, 2.1, 3 and 4.2 mm respectively. Figure E represents the voltage density and the deposition of the fibres in substrates of different height. Figure F plots the linear relationship between the height of the microfabricated substrates and the distance of the outer area that surrounds the electrospun rings.

FIG. 2 is a SEM micrograph of a section of the electrospun scaffold showing an electrospun artificial niche (A). Light microscopy micrograph (phase contrast) of electrospun scaffold (B) and detail of a microfabricated niche (B′),Image of several PLGA rings showing the structures are reproducible (C). Detail of electrospun outer ring with 1.2 cm of diameter (D).

FIG. 3A (phase contrast) shows a PLGA electrospun artificial niche with a horseshoe shape which reproduces in detail the morphology of the Palisades of Vogt in the limbus. Micrograph 3B is a SEM image of the PEGDA microfabricated niche used as an electrospun collector.

FIG. 4 is a SEM micrograph (A, B) of electrospun scaffold showing the differences in density of fibres between the inner part (A) of the scaffold and the outer ring (B). Pictures C and D are high magnification micrographs of both inner and outer areas. Figure E represents the diameters of the fibres in the thinner and thicker areas showing that there are no significant differences p<0.05 between the fibre diameters in these areas.

FIG. 5 shows A) Fluorescence image of phalloidin-TRITC-stained RLF growing on PLGA scaffold. (B) Confocal and differential interference contrast images of RLF on electrospun microfabricated U-shaped pockets. (C) The RLF on the PLGA scaffold were stained for vinculin (green), phalloidin-TRITC (red) and DAPI (blue); vinculin shows focal adhesion points between cell and substrate homogeneously distributed along the cytoplasm. Alamar blue fluorescence values at days 1, 4 and 8 showing the proliferation of RLF on PLGA rings and on PLGA analogous plain scaffolds; the samples were compared to TCP 2-D controls (no significant differences (p>0.05) were observed between the two PLGA forms).

FIG. 6 is a Schematic of the microstereolithography set-up used for the production of PEGDA rings. The laser beam was expanded using a telescopic lens arrangement. The expanded beam was consequently projected onto a computer programmable digital multimirror device (DMD), which reflects the desired image created by a user designed bitmap into a focusing lens followed by a silver-coated mirror which directs the desired image into a vial containing the photocurable polymer. The samples were cured using a two-layer model, with the first layer being the base (L1). The second layer (L2) presents the microfeatures or niches.

FIG. 7 is a Schematic of the electrospinning set-up used for the production of electrospun outer rings. (A) Electrospinning rig showing the collector made of electroplated aluminium and PEGDA microfabricated structures of different heights. (B) Drawing of the collector after the electrospinning process; SEM detail of the PLGA electrospun mats and image of an electrospun replica of the PEGDA rings containing micropockets. (C) Schematic of the parts of the electrospun rings.

FIG. 8 shows A) Phase-contrast image showing a PLGA electrospun artificial niche with a horseshoe shape which reproduces the morphology of the palisades of Vogt in the limbus. (B) High-magnification SEM image of a horseshoe niche, showing some degree of fibre alignment (orange arrows). (C) Confocal 3-D fluorescence reconstruction of a PLGA micropocket which was electrospun with 0.8 wt. % of Rhodamine; the white arrow indicates the main direction of fibre alignment. (D) Confocal and differential interference contrast images of electrospun niche and RLF stained with phalloidin-FITC; the white arrow highlights how the cells align in the same direction of the electrospun fibres.

FIG. 9 shows (A) Fluorescence image of RLE on the inner and outer parts of the PLGA scaffold. (B) RLE on a PLGA circular pocket. (C) SEM image of RLE growing on the mat of PLGA fibres. (D) High-magnification SEM image of epithelial cells showing attachment to the PLGA fibres.

FIG. 10. shows the correlation between SEM and OCT when characterising different areas of the electrospun ring with different fibres density.

FIG. 11 is a Schematic of Corneal electrospun ring. SEM lateral view of electrospun ring containing a micropocket (A). SEM image of random orientated fibres from the central part of the hybrid membrane (B). SEM high magnification image of electrospun niche with orientated fibres (C) and SEM image of area joining outer ring and niche showing aligned fibres (D).

FIG. 12 shows images of distribution of MTT staining (formazan crystals formed after incubation) on electrospun PLGA rings and plain PLGA scaffolds. Image A shows staining of cells located in 6 areas mimicking the micropockets present in the electrospun microfabricated rings; image B is a high magnification micrograph of one of the stained areas the areas (cells were 6 days in culture. Image C shows staining of 6 micropockets of an electrospun ring loaded with cells for 6 days and image D is a high magnification micrograph of one of the pockets showing cells migrating following the aligned fibres that connect the outer ring with the middle random aligned area of the membrane. Plot E shows MTT staining absorbance values after 6 days in culture (n=6, P□0.05) for RLF on plain PLGA scaffolds (white), microstructured rings (grey) and a control glass coverslip (black); no significant differences in viability were observed between rings and plain scaffolds.

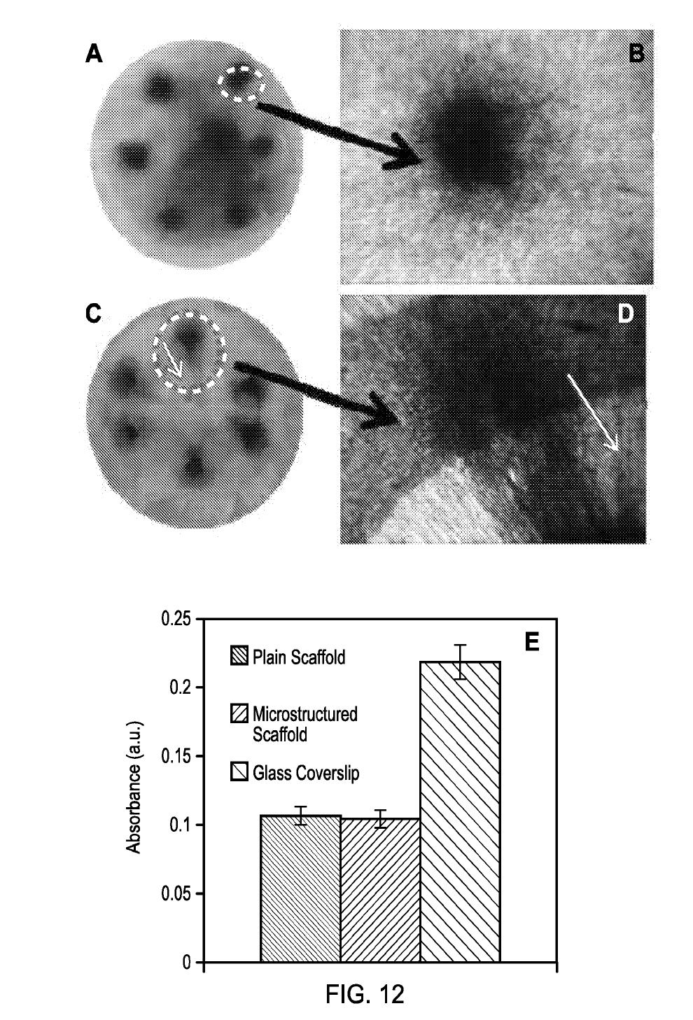

FIG. 13 shows Images A-F, which show rabbit limbal fibroblast on niches and plain scaffolds. Images G-H show rabbit limbal epithelials inside and outside the niches. Images A, B and C are fluorescence and optical images of RLF stained with Phalloidin-FITC after 6 days in culture inside a microfabricated pocket: image A is an optical image of the pocket, image B is a fluorescence image of the aligned cells inside the pocket and image C is a merged image of A and B. Images D, E and F are fluorescence and optical images of RLF stained with Phalloidin-FITC after 6 days in a plain scaffold with random fibres: image A is an optical image of the random fibres in scaffold, image B is a fluorescence image of cells on the scaffold and image C is a merged image of D and E. Image G is a confocal image showing rabbit limbal epithelial cells inside a microfabricated pocket and Image H is a rabbit limbal epithelial cell in the central part of the hybrid membrane (where the fibres are randomly organised).

FIG. 14 shows Rabbit limbal epithelial cells on PLGA rings stained for CK3 (differentiation marker) and P63 (stem cell marker) at different time points. Images A and B correspond to CK3 positive cells at 1 and 14 days of culture. Images C and D show cells positive for P63 at 1 and 14 days of culture. In image 6 a high magnification micrograph is shown highlighting the presence of the staining in the nuclei.

FIG. 15 Image A is an optical microscopy image of a fibrin-glue treated niche with a limbal explant located on it. Images B and E show cell outgrowth from the explant; image B shows Phalloidin-TRITC (red) and DAPI staining (blue) and image E is an SEM image of the cell outgrowth. Images C and F show positive staining for CK3 and P63 of cells growing out from the explants. Image C is a confocal Z-stack 3D reconstruction of an explant on an electrospun niche showing cell outgrowth; cells coming out from the explant were positive for BrdU staining indicating replicative activity. Images D and E correspond to explants kept in culture for 2 weeks. Images B, C and F correspond to explants kept 3 weeks in culture.

FIG. 16 shows H&E staining of a fresh rabbit cornea (A) and H&E staining of a tissue engineered cornea result of cell transfer from an electrospun ring to a previously wounded cornea. CK3 staining of a fresh rabbit cornea (green) using DAPI (blue) as counterstaining (C). CK3 positive staining for the tissue engineered cornea (D).

FIG. 17 shows H&E images of cell outgrowth and transfer of cells coming out of tissue limbal explants placed both facing the cornea wound bed and facing up (A, C). Immunocytochemistry images of Explants showing cell outgrowth; cells were positive in both cases for Ck3 (B, D) and for P63 (E).

### DETAILED DESCRIPTION

The inventors have surprisingly identified that microfabricated 3D structures can be engineered onto electrospun collectors as a template for electrospun scaffolds.

This allows one to produce complex shapes reproducibly. This can then be used as a template on which to spin electrospun scaffolds. Varying the height of the collector allows one to fine-tune the scaffold produced. In one embodiment, the scaffold may be designed for use in cell delivery for corneal regeneration, by providing the scaffold with artificial stem cell niches that mimic the Palisades of Vogt in the limbus. The approach may also be used for any other applications where scaffold complexity is required.

Using this approach the inventors have shown the production scaffolds with complex 3D architecture, specifically defined cavities that may function as stem cell niches, using this one-stage electrospinning approach. Advantageously, the defined cavities provide a protective microenvironment which can house cells, such as stem cells, making the scaffolds particularly useful for cell transplantation and engraftment.

The inventors have used static conditions for the development of a simple single-step method for the fabrication of hybrid scaffolds with different levels of organisation. The electrospinning fibres were organised macroscopically using the shape of the microfabricated collectors to give an electrospun outer ring of 1.2 cm diameter. At the same time fibres were also organised on top of the microfabricated features of the collector producing the electrospun cavities of 150-300 μm in diameter. This second level of organisation is important at a cellular level, with the cavities acting as potential artificial niches and providing some degree of physical protection to cultured cells added to these scaffolds.

The scaffolds of the invention have potential use as corneal implants for the delivery of cultured cells to the cornea, so as to encourage re-epithelialisation.

Preliminary cell culture of rabbit limbal fibroblasts and limbal epithelial cells on the scaffolds of the invention showed that cells retained their typical morphology when seeded onto the scaffolds and that they can be localised in the artificial niches.

The inventors have shown positive cell transfer from the ring scaffolds to damage corneas both with cell suspension and with explants. The use of the microfabricated rings with explants it is interesting from the point of view of clinical applications. The use of small pieces of tissue would allow the surgeon to take the ring-scaffold off-the-shelf offering the opportunity of placing the tissue in theatre and avoiding the need of previous cell expansion.

The results described herein demonstrate the potential of using microfabricated biodegradable membranes as epithelial cell delivery devices for corneal repair. The membranes of the invention have been shown to mimic the morphology and distribution of limbal stem cell niches in the eye. The data demonstrates that rabbit corneal cells specifically seeded in the areas of the micropockets were viable in the membranes after 6 days and no differences in viability (MTT assay) were observed between microfabricated rings and plain PLGA scaffolds. The ultimately aim of this work is the creation of niche microenvironments with self-renewing characteristics.

The data confirms that the membranes of the invention support explant outgrowth and they allowed cell transfer achieving in some cases a multi-layered epithelium very similar to the epithelium observed in fresh rabbit corneas. The possibility of using limbal explants on the microfabricated membranes would be of great benefit for surgeons. In operations where one of the eyes is damaged, surgeons can take a biopsy from the other eye and place the explants on the microfabricated membrane in theatre.

### Scaffolds

The invention provides a method for producing an electrospun scaffold, comprising electrospinning a polymer or co-polymer onto a template comprising a conductive collector having a three dimensional pattern thereon, wherein said electrospun polymer or co-polymer preferentially deposits onto said three dimensional pattern.

In addition, the invention provides an electrospun scaffold having at least one pocket therein capable of acting as a stem cell niche produced in accordance with the methods of the invention.

In addition, the invention provides an electrospun scaffold, comprising a biodegradable polymer or co-polymer, wherein said scaffold comprises at least one cavity therein capable of acting as a stem cell niche.

The term “scaffold”, as used herein, refers to any material that allows attachment of cells, preferably attachment of cells involved in wound healing. “Attachment”, “attach” or “attaches” as used herein, refers to cells that adhere directly or indirectly to a substrate as well as to cells that adhere to other cells. Preferably the scaffold is three dimensional.

The terms “electrospinning” or “electrospun,” as used herein refers to any method where materials are streamed, sprayed, sputtered, dripped, or otherwise transported in the presence of an electric field. The electrospun material can be deposited from the direction of a charged container towards a grounded target, or from a grounded container in the direction of a charged target. In particular, the term “electrospinning” means a process in which fibres are formed from a charged solution comprising at least one natural biological material, at least one synthetic polymer material, or a combination thereof by streaming the electrically charged solution through an opening or orifice towards a grounded template. As used herein, the terms “solution” and “fluid” refer to a liquid that is capable of being charged and which comprises at least one natural material, at least one synthetic polymer, or a combination thereof.

In the present invention, the polymer and/or co-polymer are electrospun onto a template. The template comprises a conductive collector having a three dimensional pattern thereon. The collector may be formed of any electrically conductive material, such as a metal. Preferably the collector is formed from aluminium, for example electroplated aluminium or an aluminium sheet, such as aluminium foil. Alternatively, the collector may be formed from an electrically conductive material comprising aluminium, brass, copper, steel, tin, nickel, titanium, silver, gold or platinum.

The three dimensional pattern may be formed on the collector using any suitable method known in the art. In one embodiment, the three dimensional pattern may be microfabricated on a surface of the collector. By way of example, the pattern may be microfabricated using microlithography, bonding, etching or injection molding. In one embodiment the pattern may be created by photolithography, microstereolithography or shadow masking. Preferably, the microfabricated three dimensional structures are microfabricated using microstereolithography, more preferably by a layer by layer photocuring approach based on the patterning of photocurable polymers, for example polyethylene glycol diacrylate.

In a preferred embodiment, the three dimensional pattern is non-conductive/insulating. Examples of non-conductive/insulating polymers, from which the three dimensional pattern may be formed include example acrylated polymers, such as polyethylene glycol diacrylate, polyethylene glycol dimethacrylate or pentaerythritol tetraacrylate. Alternatively, the pattern may be formed from Thiol-ene based polymers, or ceramics, such as ORMOCER.

Electrospinning a polymer or co-polymer on to the template having the three dimensional pattern thereon, produces a scaffold that incorporates a mirror image of the three dimensional pattern within a surface thereof.

Preferably, the three dimensional pattern is dimensioned to provide a scaffold comprising at least one cavity capable of acting as a stem cell niche. Preferably, the pattern provides a scaffold having a cavity having a diameter of from 10 μm to 500 μm, preferably from 50 μm to 400 μm, still more preferably from 150 μm to 300 μm and a depth of from 10 μm to 1000 μm, preferably a depth of from 50 μm to 150 μm. Preferably, the three dimensional pattern is dimensioned to provide a scaffold comprising multiple cavities.

Preferably, the three dimensional pattern is dimensioned to provide a scaffold of non-uniform depth.

The inventors have identified that the rate of deposition of the electrospun fibres onto the template, and hence the dimensions, i.e. the depth/density of the scaffold, is influenced by both the topography and the conductivity of the template.

The inventors have identified that electrospun fibres preferentially deposit onto the three dimensional pattern provided on the collector. This is because as the height of the pattern above the collector surface increases, the electrospun fibres have less chance of reaching the collector surface and are more likely to deposit on the three dimensional pattern. This is due to the strong effect of Coulombic interactions between the fibres and the collector (Zhang and Chang 2007; Zhang and Chang 2008)

Moreover, the inventors have surprisingly identified that electrospun fibres preferentially deposit onto a non-conductive surface, as opposed to a conductive surface. In view of this surprising observation, the inventors have provided a template comprising a non-conductive pattern on a conductive collector, thereby facilitating preferential deposition of electrospun fibres onto the pattern.

As used herein, the term “preferentially deposit” refers to an increased rate/amount of deposition onto the non-conductive surface compared to the rate/amount of deposition onto the conductive surface.

Accordingly, the inventors have identified that the depth/density of electrospun deposition onto a template may be controlled by varying both the conductivity and high of a pattern on a conductive collector. This observation has allowed the inventors to provide an improved method of electrospinning that allows the formation of scaffolds having defined cavities therein that are capable of serving as stem cell niches. Providing a scaffold with varying/non-uniform depth and density advantageously provides a scaffold having a varying rate of degradation, with deeper/denser areas degrading less rapidly than less deep/less dense areas.

There are many factors involved in the electrospinning process which may affect scaffolds fibre diameter and pore size. The key variables are solution viscosity, surface tension, and viscoelasticity of the spinning solution. These are directly related to the concentration of, and molecular weight of the polymer, as well as the solvent used. The dielectric properties of the solution also play a key role (Kowalczyk et al., Biomacromolecules. 2008 July;9(7):2087-90; Thompson et al., J. Polymer. 2007;48:6913-6922; Mitchell and Sanders, J Biomed Mater Res A. 2006 July;78(1):110-20).

Another source of variation in electrospinning which is perhaps not well documented is that once the polymer is in solution it can change or degrade on storage and the same concentration of polymer does not always yield a solution with the same viscosity. Thus the molecular weight of the polymer will decrease rapidly over time (particularly the 50/50 PLGA). Therefore, fresh polymer is preferably used for spinning.

The electrospun polymer may be a co-polymer. The term “co-polymer” as used herein is intended to encompass co-polymers, ter-polymers, and higher order multiple polymer compositions formed by block, graph or random combination of polymeric components.

The properties of the electrospun materials can be adjusted in accordance with the needs and specifications of the cells to be suspended and grown within them. The porosity, for instance, can be varied in accordance with the method of making the electrospun materials matrix.

As used herein, a natural biological polymer material can be a naturally occurring organic material including any material naturally found in the body of a mammal, plant, or other organism. A synthetic polymer material can be any material prepared through a method of artificial synthesis, processing, or manufacture. Both the biological and polymeric materials are capable of being charged under an electric field.

Suitable naturally occurring materials include, but are not limited to, amino acids, polypeptides, denatured peptides such as gelatin from denatured collagen, carbohydrates, lipids, nucleic acids, glycoproteins, lipoproteins, glycolipids, glycosaminoglycans, and proteoglycans. In one embodiment the naturally occurring material is an extracellular matrix material, for example collagen, fibrin, elastin, laminin, fibronectin, heparin, fibrinogen. Such extracellular matrix material may be isolated from cells, such as mammalian cells, for example of human origin. Preferably the naturally occurring material is collagen. Alternatively, the naturally occurring polymer is chitin.

Preferably the polymer is a biodegradable polymer. As used herein, the term “biodegradable” refers to material or polymer that can be degraded, preferably adsorbed and degraded in a patient's body. Preferably the scaffold is biodegradable, i.e. is formed of biodegradable materials, such as biodegradable polymers naturally occurring biological material. Examples of suitable biodegradable materials include, but are not limited to collagen, poly(alpha esters) such as poly(lactate acid), poly(glycolic acid), polyorthoesters, polyanhydrides polyglycolic acid and polyglactin, and copolymers thereof.

Other suitable biodegradable polymers include cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or thereof.

As used herein the term “degradation” relates to the breakdown of the polymer structure of the scaffold. This breakdown of structural integrity is accompanied by the release from the scaffold of degradation products from the polymer and a reduction in the mechanical strength of the scaffold.

More preferably, the polymer is biocompatible. As used herein, the terms “biocompatible” and “biologically compatible” are used interchangeably to the ability of a material, i.e. a polymer, to be implanted into or be administered to a human or animal body, without eliciting any undesirable local or systemic effects in the recipient, for example, without eliciting significant inflammation and/or acute rejection of the polymer by the immune system, for instance, via a T-cell response.

Preferably the scaffold comprises a biocompatible synthetic polymer. Examples of biocompatible synthetic polymers include, but are not limited to, poly(urethanes), poly(siloxanes) or silicones, poly(ethylene), polyvinyl pyrrolidone), poly(2-hydroxy ethyl methacrylate), poly(N-vinyl pyrrolidone), poly(methyl methacrylate), polyvinyl alcohol) (PVA), poly(acrylic acid), polyvinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate), polyvinylhydroxide, poly(ethylene oxide) (PEO) and polyorthoesters or co-polymers thereof thereof Preferably, the biocompatible polymer is PGLA.

Preferably the polymer is poly(methyl methacrylate) (PMMA) or poly(hydroxy ethyl methacrylate) HEMA.

Alternatively, the polymer is poly(c-caprolactone), poly(DTE carbonate), poly(propylene carbonate), poly(L-lactic acid).

Preferably, the co-polymer is poly(L-lactic-co-c-caprolactone), poly(ethylene glycol-co-lactide), poly(D,L-lactide-co-glycolide), Poly(ethylene-co-vinyl alcohol), Poly(D,L-lactic-co-glycolic acid) and PLGA-B-PEG-NH2, Poly(D,L-lactic-co-glycolide), collagen and elastin, poly(L-lactic-co-ε-caprolactone) and collagen, or poly(L-lactic acid) and hydroxylapitate.

Most preferably, the co-polymer is poly(lactic-co-glycolic acid).

The invention includes scaffolds comprising a combination of synthetic polymers and naturally occurring biological material, for example a combination of collagen and PLGA. The relative amounts of the synthetic polymers and naturally occurring biological material in the matrix can be tailored to specific applications

Preferably the electrospun material is a polylactide, or a derivative thereof. Alternatively, the electrospun material is polyurethane, preferably polyurethane based on hexamethylene diane and polylactide derivatives. Alternatively the electrospun material is a chitosan derived material.

Preferably, the electrospun scaffold is functionalized, for example, by the addition of passive or active agents such as additional therapeutic or biological agents.

As used herein, the term “stem cell niche” refers to a cavity within an electrospun scaffold capable of housing one or more cells, e.g. stem cells, therein and providing a sheltering environment that physically protects said cells from physical disturbance and/or from stimulus that may promote differentiation and apoptosis. Preferably, the niche is a cavity defined by a concave surface within an electrospun scaffold, for example in the form of a pocket, a recess, a groove or a ridge.

Preferably, the cavity has a diameter of from 10 μm to 500 μm, preferably from 50 μm to 400 μm, still more preferably from 150 μm to 300 μm and a depth of from 10 μm to 1000 μm, preferably from 50 μm to 150 μm.

Preferably, the scaffold comprises multiple cavities, for example at least 5, 10 15, 20, 50, 100, 200 or 500 cavities.

Preferably the scaffold has a non-uniform depth. For example, a scaffold for ocular implantation may contain an outer ring, having a first depth, which comprises at least one stem cell niche, which in situ is capable of acting as an artificial limbus, each niche acting as a protected stem cell reservoir. The scaffold may also contain an inner area, having a second depth which is less than said first depth, which serves as a scaffold for stem cells to move over the denuded cornea for corneal regeneration. This relatively thinner depth of the inner area results in a high rate of degradation leaving the cells in place on the cornea.

Preferably the cavity comprises at least one cell, for example an epithelial cell, such as a limbal or dermal epithelial cell. Preferably said cell is a stem cell, for example an epithelial stem cell, i.e. a cell capable of differentiating into an epithelial cell. More preferably the epithelial stem cell is a corneal epithelial stem cell, a skin epithelial stem cell, a buccal musoca epithelial stem cell, an oesophageal epithelial stem cell, an intestinal epithelial stem cell, a vaginal epithelial stem cell, a urethral epithelial stem cell, a respiratory epithelial stem cell or a bladder epithelial stem cell. Still more preferably, the stem cell is a corneal epithelial stem cell or a limbal epithelial stem cell. Alternatively, the stem cell is a mesenchymal stem cell.

In one embodiment the cavity may further comprises at least one cell that promotes maintenance of the stem cell, for example specialised support cell for the cornea or any mesenchyme derived stromal cell e.g. fibroblasts, such as fibroblasts from skin or oral mucosa or any other epitheial tissue.

The aforementioned cells may be seeded into the cavity by any technique known in the art. For example, the cells may be pipette into the cavity.

In one embodiment, the cavity may further comprise any extracellular matrix component such as fibronectin, vitronectin, collagen, laminin. Also circulating materials involved in wound healing such as fibrin (formed during clot formation and a natural adhesive) and heparin (secreted during wound healing and able to bind and immobilise short-lived growth factors which are subsequently slowly released to promote local cell migration and proliferation). The cavity may also comprise growth factors and/or short protein fragments

### Uses

Accordingly, the electrospun scaffolds of the invention are of particular use in various therapeutic settings. In particular, the scaffolds are of particular use in stem cell transplant and engraftment, as they provide a sheltering environment that physically protects said stem cells from physical disturbance and/or from stimulus that may promote differentiation and apoptosis.

Accordingly, the invention provides the use of the electrospun scaffolds of the invention as a medicament.

The scaffolds of the invention may be used to deliver cells to a tissue in need thereof. In one embodiment, the scaffolds are used to deliver corneal or limbal epithelial cells to the eye of a mammalian subject. Alternatively, the scaffolds of the invention may be used to deliver dermal epithelial stem cells to a wound bed of a mammalian subject in need thereof.

The invention provides a method of treating an ocular injury comprising implanting an electrospun scaffold of the invention into the eye of a mammalian subject in need thereof.

As used herein, the term “ocular injury” refers to conditions resulting in an insufficient stromal micro-environment to support stem cell function, for example aniridia, keratitis, neurotrophic keratopathy, Keratoconus, Meesman's dystrophy, Epithelial Basement Membrane Dystrophy and chronic limbitis; or conditions that destroy limbal stem cells such as Partial limbal stem cell deficiency, Total stem cell deficiency , Ocular herpes, chemical or thermal injuries, Stevens- Johnson syndrome, ocular cicatricial pemphigoid, contact lens wear, or microbial infection.

In one embodiment that scaffold cavity/cavities are seeded with stem cells prior to implantation. Alternatively, the scaffolds cavity/cavities may be seeded with stem cells after implantation. Preferably, said cells are autologous, i.e. said cells are derived from the individual to be treated or alternatively the cells may be non-autologous.

There is also provided an electrospun scaffold of the invention for use in treating an ocular injury.

Also provided is a method of corneal replacement, comprising implanting an electrospun scaffold of the invention into the eye of a mammalian subject in need thereof. There is also provided an electrospun scaffold of the invention for use in corneal replacement.

Also provided is a method of treating a skin wound comprising implanting an electrospun scaffold of the invention into the skin, skin wound or skin wound bed of a mammalian subject in need thereof. The method is of particular use in skin re-epithelialisation. The term “re-epithelialisation” relates to the repair, replacement, functional recovery and ultimate regeneration of damaged epithelium inside the body (including skin), or outside the body.

As used herein the term wound relates to damaged tissues, preferably damaged skin, where the integrity of the skin or tissue is disrupted as a result from i.e. external force, bad health status, aging, exposure to sunlight, heat or chemical reaction or as a result from damage by internal physiological processes. Wounds where the epidermis is damaged are considered an open wound. Wound healing is the process of regenerating the covering cell layers of a tissue, preferably by re-epithelialisation or reconstruction.

There is also provided an electrospun scaffold of the invention for use in treating a skin wound.

The invention also provides a pharmaceutical composition comprising an electrospun scaffold in accordance with the invention together with a pharmaceutically acceptable excipient, dilutent or carrier. In one embodiment the composition further comprises one or more of the following: growth factors, lipids, genes, etc., or compounds for altering the acidity/alkalinity (pH) of the wound, or compounds for altering the growth and performance of the transplanted cells and those at the margins of the wound/injury.

The term “pharmaceutically-acceptable carrier” as used herein means one or more compatible solid or liquid fillers, diluents or encapsulating substances that are suitable for administration into a human. When administered, the pharmaceutical compositions of the present invention are administered in pharmaceutically acceptable preparations. Such preparations may routinely contain pharmaceutically acceptable concentrations of salt, buffering agents, preservatives, compatible carriers, cytokines and optionally other therapeutic agents, preferably agents for use in wound healing such as growth factors, peptides, proteolytic inhibitors, extracellular matrix components, steroids and cytokines. The term “carrier” denotes an organic or inorganic ingredient, natural or synthetic, with which the active ingredient is combined to facilitate the application. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. As used herein, a pharmaceutically acceptable carrier includes any conventional carrier, such as those described in Remington's Pharmaceutical Sciences, by E. W. Martin, Mack Publishing Co, Easton, Pa., 15th Edition (1975).

In a further aspect there is provided a pharmaceutical composition in accordance with the invention for use as a medicament, for example, for use in treating ocular injury, corneal replacement or wound healing.

The compositions or electrospun scaffolds of the invention are administered/for administration in effective amounts. An “effective amount” is the amount of a composition or electrospun scaffolds that alone, or together with further doses, produces the desired response. The compositions or electrospun scaffolds used in the foregoing methods preferably are sterile and contain an effective amount of the active ingredient for producing the desired response in a unit of weight or volume suitable for administration to a patient. The response can, for example, be measured by measuring the physiological effects of the composition or micro-organ cell composites upon the rate of or extent of wound healing or corneal repair.

### EXAMPLES

### Materials and Methods

### 1.1. Fabrication of Biodegradable Electrospun Outer Rings

Electrospun outer rings were fabricated via a combination of microstereolithography and electrospinning. First, polyethylene glycol diacrylate (PEGDA) scaffolds were microfabricated using an in-house projection microstereolithography system based on a 473 nm wavelength laser and a digital micromirror device. Subsequently, the electrospinning collectors were produced by attaching the microfabricated rings onto electroplated aluminium sheets. During the electrospinning process, the fibres were preferentially deposited onto the cured PEGDA constructs adopting the shape of the underlying rings, resulting in the production of positive electrospun replicas.

### 1.1.1. Electrospinning Conditions

PLGA (50/50 DL-lactide (52 mol %): glycolide (48 mol %), 44 Kg/mol) was purchased from Purac Biomaterials. The copolymer was dissolved in DCM at 25% w:w concentration and stirred 2 hours before use. The polymer was then electrospun using 4 insulin syringes at 30 μl/min rate and at voltages ranging from 10 to 13.5 Kv. The deposition time was 2 hours for all the samples.

### 1.1.2. Design of the Collectors

Polyethylene glycol structures were fabricated using microstereolithography, a layer by layer photopolymerisation method.

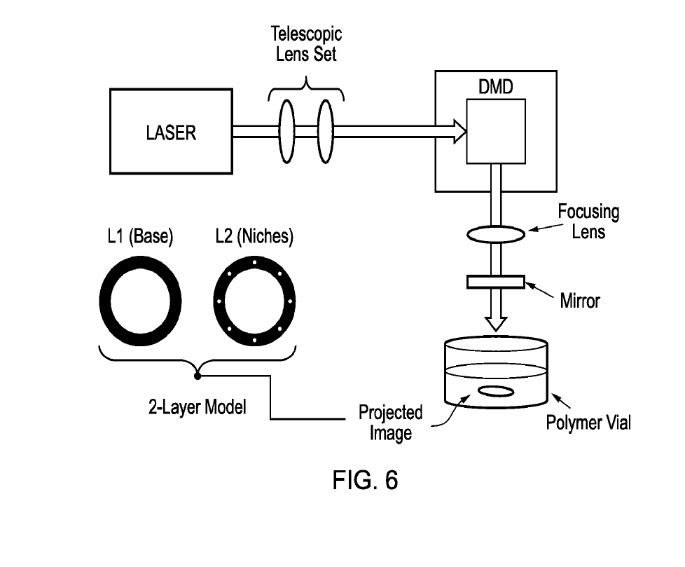

The Camphorquinone (photoinitiator, Aldrich) was mixed with the photocurable polymer (polyethylene glycol diacrylate, Mₙ 258, Aldrich) in a percentage 1% w/w; the mixture was stirred for 10 min. The polymer was then irradiated with a blue laser (MBL-III 473 nm; 150 mW). The laser beam was focused onto a multimirror device (DMD) that receives information of an attached CPU and reproduces the image with the desired shape. The DMD was followed by a mirror which directs the created image into the polymer. The use of a layer-by-layer technique allows the fabrication of complicated structures giving the possibility of designing different niche morphologies. The light reflected from the DMD is collected by a 2.5 cm diameter 10 cm focal length lens (Thorlabs) and the image is projected by a mirror into the sample vial containing the photocurable polymer (FIG. 6) The vial is positioned on a 1-D translation stage, which allows the fabrication of complicated structures via layer-by-layer structuring. This optical set-up provides the possibility of designing 1.5 cm diameter objects with a minimum resolution of 50 lm, thus enabling the construction of macroscopic ring-shaped objects with different niche morphologies.

The sample was cured using a simple design based on a 2-layer model (FIG. 6), the first layer being the base. The second layer contained the artificial niches or micropockets in a number of 6-8 and ranging sizes from 150 to 300 μm. Different morphologies of artificial niches were developed by adjusting the designs of the layers. The optimized time of curing was 60 s for each layer.

PEGDA collectors (non-conductive) were placed on electroplated aluminium sheets (conductive) previously sterilised with methanol. The whole assembly was sterilized before spinning with Azo Wipes (70% Isopropanol Hard Surface Bactericidal Wipes). The surface was carefully wiped three times and left for 1 h to air-dry in a fume cupboard located in a cleanroom.

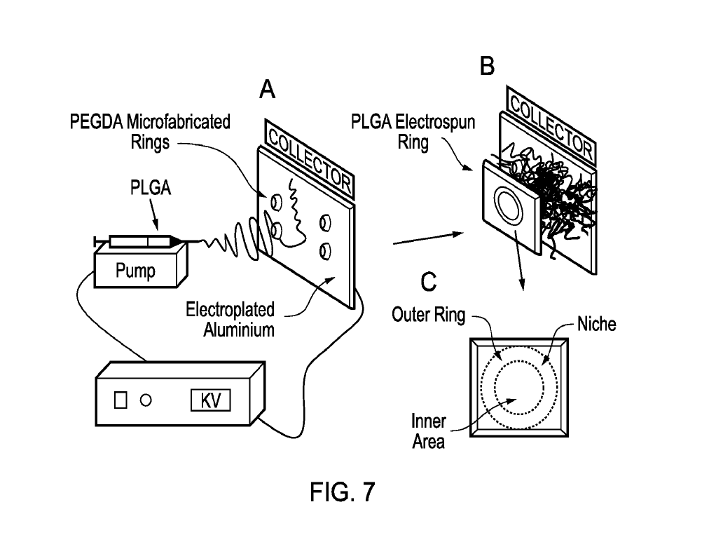

The electrospun fibres reached the polymeric vertical static collectors replicating the shape of the microfabricated substrates resulting in the production of electrospun outer rings with electrospun micropockets (FIG. 7).

### 1.2. Morphology of the Scaffolds

The electrospun outer rings consisted of two main parts: (i) an outer ring containing microfeatures, which was the direct positive replica of the PEGDA ring; and (ii) an inner area or central part, which was in direct contact with the metallic collector (FIG. 7C).

The morphology of the scaffolds was explored with both phase contrast and electron scanning microscopy (SEM). The shape and size of the microfabricated niches was studied using the same techniques.

Phase Contrast Microscopy was carried out using an inverted Olympus CK40 microscope. For SEM observation a Philips X-L 20 microscope was used; the samples were sputter-coated with gold for 3 minutes in anemscope SC 500 coater.

### 1.3. Study of the Height of the Templates

The appearance and morphology of the scaffolds varied according to the height of the electrospinning template. The height of the template was varied from 0.9 to 4.2 mm. Greater template height resulted in the formation of hybrid scaffolds (inner low density area and outer high density area) that will be described in the results section.

### 1.4. Fibre Diameter and Thickness of the Scaffold

Fibre diameter was calculated from SEM images both in the inner part and the outer part of the scaffolds. A total of 10 fibres were measured per image with a total number of 10 images.

The thickness of the scaffolds was measured using a micrometer.

Two different samples were measured in 3 different areas. For the measurements of the inner area, the materials were placed between two coverslips and the thickness was calculated as the difference.

### 1.5 Characterization of PLGA Micropockets

The structure of the PLGA micropockets was characterized using different techniques: SEM, confocal imaging and optical coherence tomography (OCT).

For confocal imaging, Rhodamine-loaded scaffolds were prepared by adding Rhodamine (0.8 wt. %) to the spinning solution. The spinning procedure was performed as described in Section 2.1. After spinning, z-stacks of the scaffolds in the areas of the micropockets were taken and 3-D reconstructions of those areas were performed using ImageJ software.

SEM observations were performed using a Philips X-L 20 microscope, as described above; additionally, the niches were imaged by SEM from different perspectives by rotating the samples from 15 to 70. The micropockets were also imaged by an OCT system with a laser source (Santec HSL-2000) operated at 10 kHz rate, with 10 mW output power and a central wavelength of 1300 nm.

### 1.6. Cell Culture

This study shows preliminary cell culture work on biodegradable outer rings using primary rabbit limbal fibroblasts and primary rabbit limbal epithelial cells. Rabbit eyes were obtained from the pet food company Woldsway Foods Ltd., Spilsby, UK.

Rabbit limbal fibroblasts were isolated from stromal tissue. The cells were explanted and cultured in DMEM containing 10% fetal bovine serum, 1% penicillin/streptomycin and 1% L-Glutamine. Fibroblasts were used up to maximum passage of 7.

Primary rabbit limbal epithelial cells were isolated as follows. The limbal area was separated from the rest of the cornea and cut it into segments which were immersed into 2.5 U/ml Dispase II solution for 45 min at 37° C. The cells were then scraped using a dissection microscope and they were collected in phosphate buffer saline (PBS). The cells were then spun down at 1000 rpm for 5 minutes, resuspended in culture medium and seeded into a T 25 flask containing irradiated 3T3s. The rabbit limbal epithelial cells were cultured in 1:1 DEMEM+Glutamax: Ham's F12, 10% fetal bovine serum,1% penicillin/streptomycin, 1% Amphotericin, 25 μl 10 ng/ml of EGF and 0.5% of insulin. Rabbit limbal epithelial cells were used up to passage 4.

### 1.7. Cell Imaging

Cells were imaged using SEM, fluorescence and confocal microscopy. For fluorescence imaging rabbit limbal fibroblasts were seeded on the scaffolds at a concentration of 8×10⁴cells/well and 25×10⁴ and stained with phalloidin-TRITC or phalloidin-FITC (to label actin filaments). Cells were fixed in 10% formalin in PBS during 30 min at room temperature; afterwards, phalloidin was added 1:1000 (palloidin-TRITC), 1:500 (phalloidin FITC) in PBS during 30 min. Cells were observed under confocal scanning microscope (Carl Zeiss LSM510-META, Germany) and under fluorescence microscopy (ImageXpress, Axon Instruments).

RLF were stained using monoclonal anti-vinculin produced in mouse (Sigma-Aldrich). Cells were seeded at a concentration of 5×10⁴ cells per sample for 7 days. After fixation with paraformaldehyde, the samples were permeabilized with 0.5% Triton X-100 for 20 min. Cells were then washed three times with PBS for 10 min and blocked with 10% goat serum (Sigma-Aldrich) for 60 min, followed by incubation with primary antibody diluted 1:150 in 1% goat serum for 60 min. Samples were washed with PBS (3 for 10 min), incubated with biotinylated secondary antimouse antibody (1:1000 in 1% goat serum, Aldrich) for 60 min at room temperature and further washed with PBS. Finally, they were incubated with tertiary antibody FITC-streptavidin (1:100 in 1% goat serum, Aldrich) for 30 min at room temperature. Cells were also treated with the nuclear staining 4′,6-diamidino-2-phenylindole dihydrochloride (DAPI) and phalloidin-TRITC.

For SEM imaging cells were washed with 0.1 M sodium phosphate buffer (PBS) and were fixed in glutaraldehyde for 1 hour then the cells were dehydrated with increasing concentration gradients of ethanol (from 35% to 100%). Finally, the samples were dried using hexamethyldisilazane (HMDS): ethanol 1:1 for 1 h and followed by 100% HMDS for 5 min (Bray, Bagu et al. 1993). After drying, coverslips were sputter-coated with gold (emscope SC 500 coater).and analysed by a Philips X-L 20 microscope.

### 1.8 Cell Viability and Proliferation

RLF were used for the study of cell viability and proliferation on the PLGA rings. An Alamar blue test (Resazurin test) was performed and triplicate samples were run for every experiment. Cells were seeded at a concentration of 5×10⁴ cell per sample and fluorescence measurements were taken at 1, 4 and 8 days. Cell viability and proliferation in the rings were compared to 2-D tissue culture plastic (TCP) controls and plain sheets of PLGA electrospun scaffolds (non-structured PLGA membranes).

For each time point, cells were gently washed with PBS and the Resazurin solution (5 μg ml⁻¹ in serum-free medium) was added for 4 h. Three aliquots of 100 μl were taken from each sample and fluorescence measurements were taken using a FLx 800 microplate fluorescence reader (Bio-Tek Instruments) at λ_{excitation}=540 nm/λₑₘᵢₛₛᵢₒₙ=635 nm. After fluorescence measurements, the Resazurin solution was removed, the cells were washed with PBS, fresh medium was added and the samples were kept in the incubator.

### 2. Results

### 2.1. Morphology of the Outer Rings and Importance of the Height of the Collector

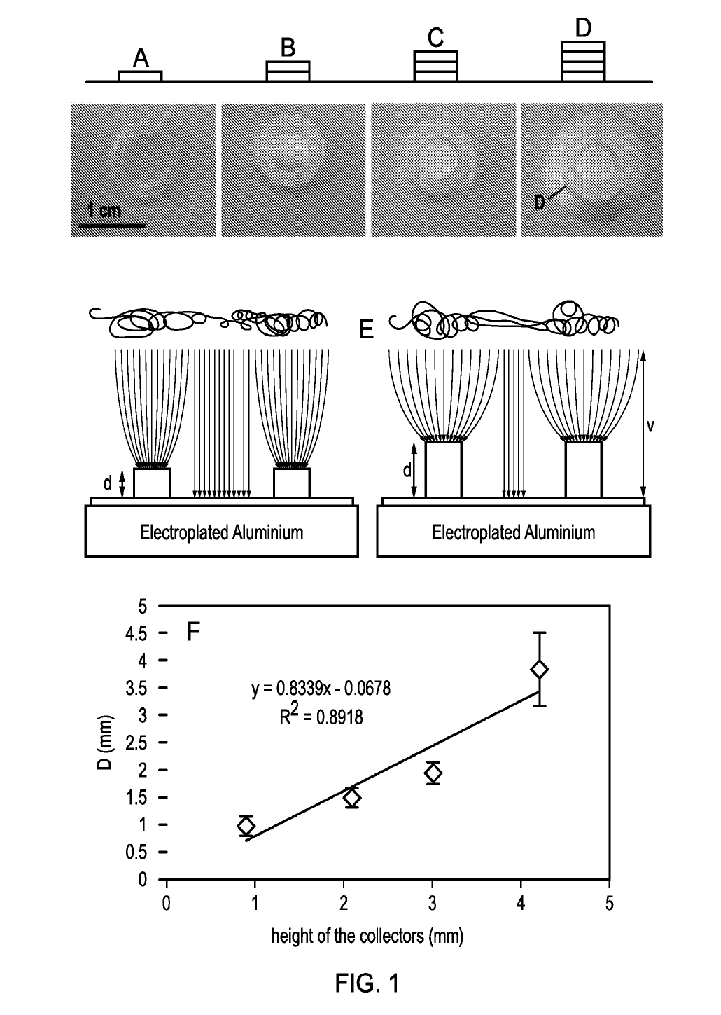

The materials were prepared using substrates of different heights ranging from 0.9 to 4.2 mm (see FIG. 1). Two kind of scaffolds were obtained: for electrospun scaffolds deposited on collectors of 0.9 cm, the thickness of the outer ring (with micropockets (artificial stem cell niches)) and the inner area was virtually the same; on the contrary, when the height of the collectors was increased the inner area became thinner resulting in hybrid structures with a thin inner area and a thick outer ring containing electrospun micropockets (artificial stem cell niches). These differences in thickness may be of interest for future applications as there will be different rates of degradation shown in these areas (thinner areas will degrade more rapidly than thicker areas).

As highlighted in FIG. 1E, the 3-D object electrically shields the deposition on the collector. When the 3-D structures (PEGDA rings) are taller, the fibres experience a greater focusing towards the raised areas, resulting in thinner electrospun mats in the non-raised areas. This enables an excellent level of control on introducing patterns of different thickness within the electrospun fibre mats, via electrospinning on objects of different heights (FIG. 1A).

Furthermore, when the height of the collector was increased, the area surrounding the thick outer rings became thinner, almost linearly (FIG. 1F).

As shown in FIG. 1 the electrospun outer rings presented a high density of fibres while the inner area of the scaffolds presented a lower density. This is observed in scaffolds prepared in collectors higher than 0.9 mm. The differences in density were evident in both SEM and phase contrast microscopy.

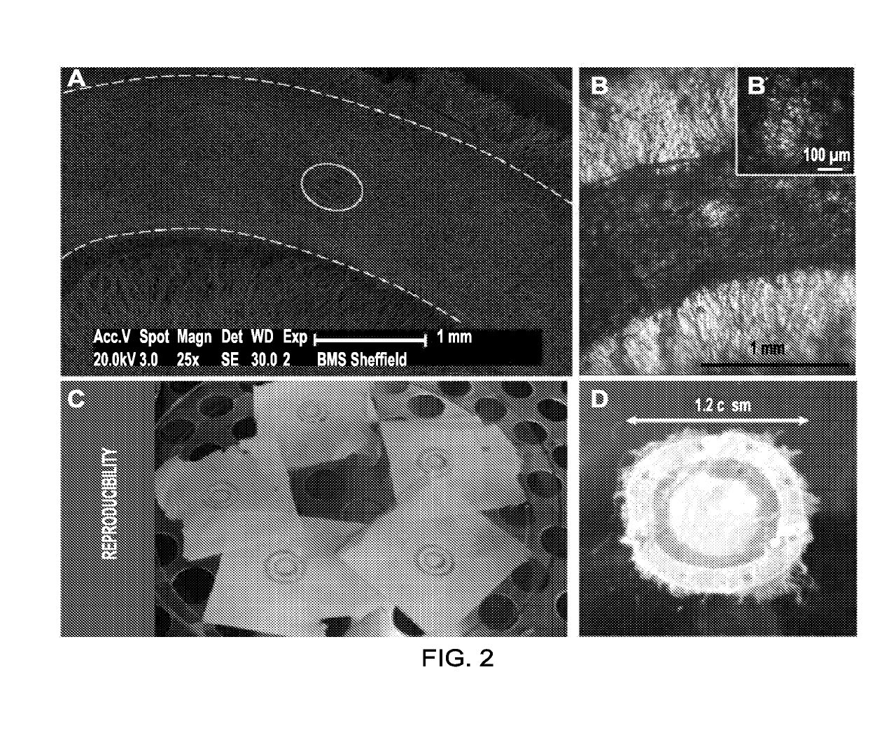

Regarding the micropockets (artificial stem cell niches) situated in the outer rings, there was also a difference in the density of the fibres in these micropockets. This can be seen to a slight extent using SEM, see FIG. 2A, but is clearly more evident when using light microscopy, FIG. 2B.

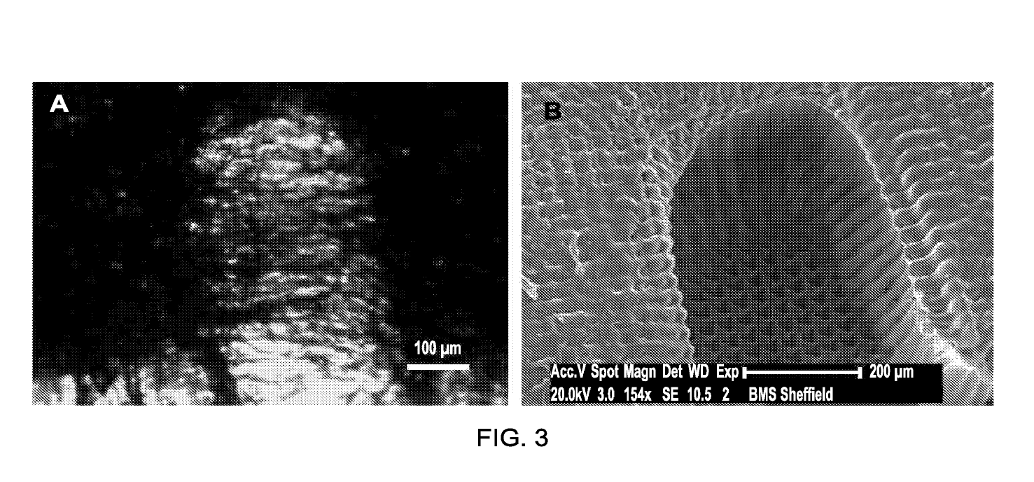

Microstereolithography is a versatile technique that allows the design of different micropocket morphologies and sizes that can be reproduced by electrospinning as shown in FIG. 3.

The morphology shown in FIG. 3 reproduces closely the curvature and shape of the palisades of Vogt in the limbus and also provides the outer ring with a micrometer open structure that would provide a clear route for migration of cells to the cornea.

### 2.3. Fibre Diameter and Thickness

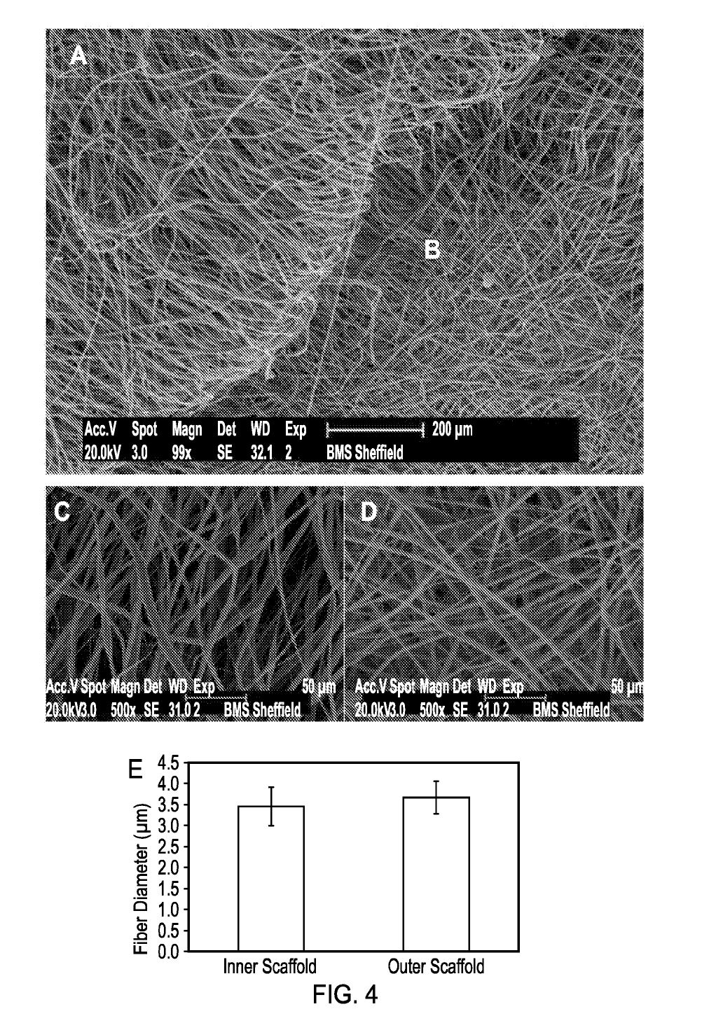

The density of the fibres per mm square was evidently lower as seen by eye in the inner area of the hybrid materials as shown in FIG. 4C and 4D) although the diameter of the fibres in both areas was identical (as shown in FIG. 4E).

No significant differences were observed between the fibre diameters of the inner and outer parts of the scaffold (p>0.05, Student's t-test).

The thickness of the electrospun outer ring was 0.36±0.01 mm and that of the inner area ranged from 0.05±0.01 to 0.12±0.01 mm, depending on the height of the collectors.

### 2.4 Niche Formation and Characterization

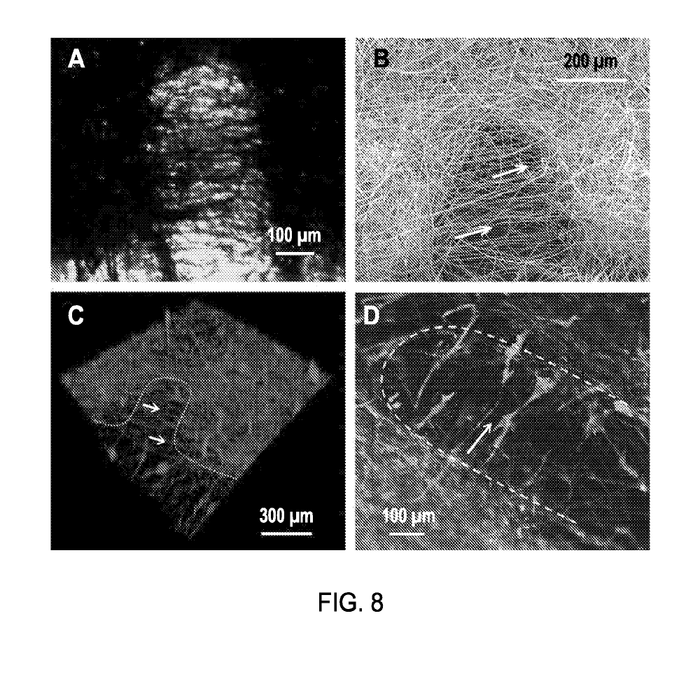

The analysis of the results obtained with imaging techniques such as OCT, SEM and 3-D confocal reconstructions allowed us to describe the electrospun microfeatures as confined areas on the micrometre scale with lower fibre density than the surrounded ring. SEM images taken from different angles allowed us to corroborate that the niches were cavities consisting of an area with low fibre density. OCT (see supporting information) also supported this fact and allowed us to measure the depth of the cavity, which varied between 100 and 180 lm. Furthermore, it is noted that the fibres that form the niche are not randomly aligned, but present a certain degree of alignment, as highlighted in FIG. 8B-D.

### 2.5. Ability of Scaffolds to Support Cell Attachment

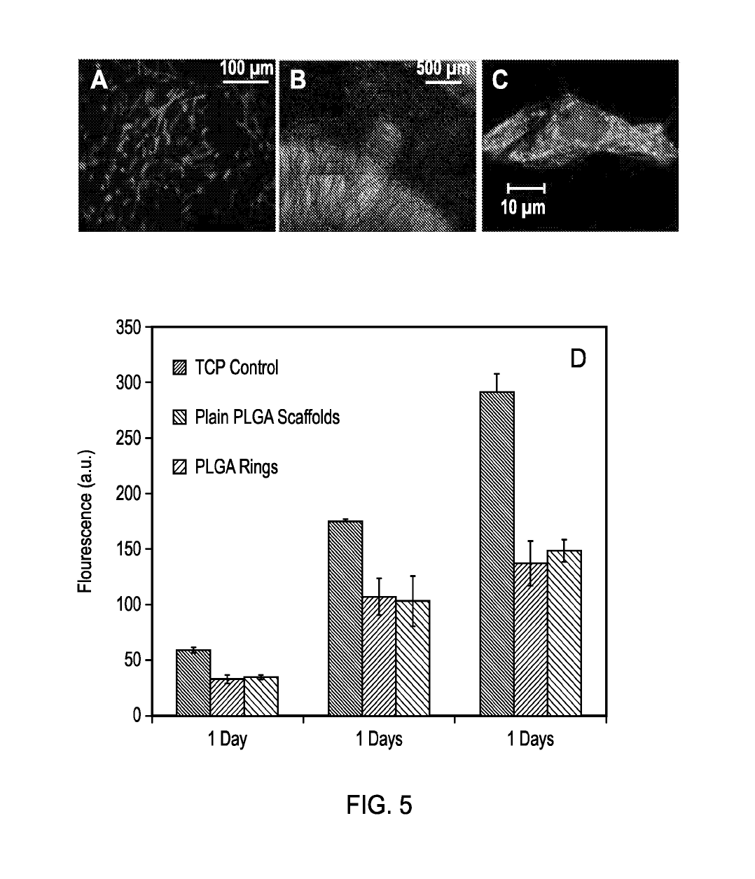

Firstly, rabbit limbal fibroblasts (RFL) were seeded onto the electrospun scaffolds and when stained with Phalloidin-TRITC showed the typical elongated morphology of fibroblasts. Phalloidin staining imaged with fluorescence (FIG. 5A) showed that the F-actin filaments of the cytoskeleton were labelled.

Confocal images were taken and merged with a differential interference contrast image. Merging these two channels showed that the cells were in the scaffold and also in the electrospun micropockets (see FIG. 5B).

In order to study the ability of RLF to attach to the scaffold, we used vinculin staining. Cells were labelled for both vinculin (red) and actin (green) using DAPI as a counter stain. Cells were positive for vinculin staining, showing multiple adhesion points uniformly distributed thorough the cytoplasm (FIG. 5C).

The viability and proliferation of RLF were studied using Alamar blue (FIG. 5D). No significant differences at 1, 4 and 8 days were observed between the PLGA rings and their analogous PLGA plain scaffolds (p>0.05, Student's t-test). Significant differences were observed between both the PLGA forms and the TCP controls, the proliferation rate being faster for the cells growing on the TCP.

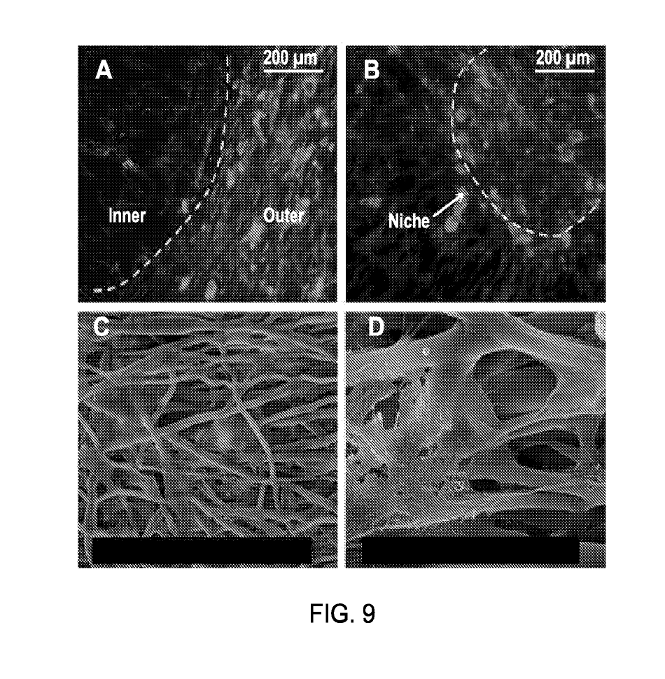

Secondly, primary rabbit limbal epithelial (RLE) cells were seeded onto the outer rings and they were stained in this case using Phalloidin-FITC and DAPI. The cells grew in both outer and inner areas of the scaffolds and also in the artificial niches (see FIGS. 9A and 9B). Moreover, samples with limbal epithelial cells (RLE) were fixed for SEM imaging showing their affinity for attaching to the PLGA scaffolds (see FIGS. 9C and 9D).

### 3. Cornea Model

### 3.1. Fabrication of Biodegradable Electrospun Outer Rings

The electrospun ring scaffolds were fabricated as described above. Specifically, the constructs were fabricated by a combination of microstereolithography and electrospinning techniques. Polyethylene dyacrilate templates were custom-designed and cured using an in-house microstereolithography device equipped with a laser emitting in the blue region of the spectrum (MBL-III 473 nm; 150 mW). The PEGDA templates were created between sizes of 1.2 and 1.6 cm in diameter and 1 mm in height; the structures were equipped with microfabricated pockets in horseshoe shape of sizes of 300-500 μm. The PEGDA rings were then placed on electroplated aluminium sheets and attached using conductive tape. PLGA (50/50 DL-lactide (52 mol %): glycolide (48 mol %), 44 kg/mol, Purac) was then spun onto the hybrid collectors. PLGA was dissolved in dichloromethane (DCM) at 20% w:w concentration and stirred for 2 hours before use. The polymer was then electrospun using four 5 ml insulin syringes at 30 μL /min rate and at voltages ranging from 12 to 15 kV. The deposition time was 1.5 hours and the distance between the needles and the collector was 17 cm.

### 3.2. Cell Isolation and Rabbit Limbal Explant Dissection

Rabbit limbal fibroblasts (RLF) and primary rabbit limbal epithelial cells (RLE) were isolated from rabbit eyes (obtained from Alison Wilson, Hook Farm, UK). For the isolation of primary rabbit limbal epithelial cells the limbal region was separated from the rest of the cornea and then cut into segments under a dissection microscope.

For the isolation of rabbit limbal explants those segments were disinfected in iodine for 1 min and after they were cut into small pieces (100-500 μm) with a scalpel. For cell isolation the segments were immersed in 2.5 U/ml Dispase II solution for one hour at 37° C. Epithelial cells were then scraped, collected in media and spun down at 1000 rpm for 5 minutes; after the cells were seeded into a T 25 flask containing irradiated 3T3s. The rabbit limbal epithelial cells were cultured in 1:1 DMEM+Glutamax: Ham's F12, 10% fetal bovine serum, 1 U/ml penicillin, 100 mg/ml streptomycin, 2.5 μg/ml amphotericin, 10 ng/ml of EGF and 5 μg/ml of insulin. RLE cells were used at passage 1. Rabbit limbal fibroblasts RLF were isolated from stromal tissue remaining after isolation of RLE and they were cultured in DMEM containing 10% fetal bovine serum, 1U/m1 penicillin, 100 mg/ml penicillin/streptomycin, 2 mM L-glutamine and 0.625 mg/ml amphotericin. RLF were used between passages 4 and 7.

### 3.3. Ex vivo 3D Cornea Model

For the setting up of the 3D corneal models, rabbit eyes were first disinfected using 3% videne antiseptic solution (Ecolab) and then immersed into 0.14% ammonium hydroxide (Sigma Aldrich) during 5 minutes followed by washing with PBS. The epithelium in both the central cornea and the limbal region was then removed by scraping using a sclerotome knife. The corneas were mechanically supported by a combination of 0.5% agar (Sigma Aldrich) and 5 mg/ml collagen from rat tail (Fluka). The corneas were cultured in the epithelial culture medium described above. Positive and negative controls of the denuded corneas were also maintained in culture for the same periods of time the negative controls confirming the lack of formation of a new epithelium.

For the transfer of cultured cells, ring scaffolds of scaffolds of 1.2 and 1.6 mm in diameter were mechanically supported by cell 6-well plate cell crowns and rabbit limbal epithelial cells were then seeded in the area of the pockets as described above for the viability assay. Cells were kept in culture overnight and placed on the organ model the following day. For the transfer of rabbit limbal explants, the pieces of limbus were directly placed on the ring scaffolds; using a dissecting microscope the explants were placed directly on the niches and also in the center of the hybrid membrane which was previously treated with fibrin glue. The scaffolds with either with cells or explants were then placed on the deliberately denuded corneas using different conditions: cells facing up/cells facing down and air-liquid interface/submerged. The organ culture models were kept in culture for 4 weeks and after the corneas were fixed using 3.7% formaldehyde and processed for conventional histology to produce 6 μm paraffin sections (Microtome Leica RM 2145) and then stained with haematoxylin and eosin (H & E).

### 3.4. Characterisation of Ring Scaffold Complexity

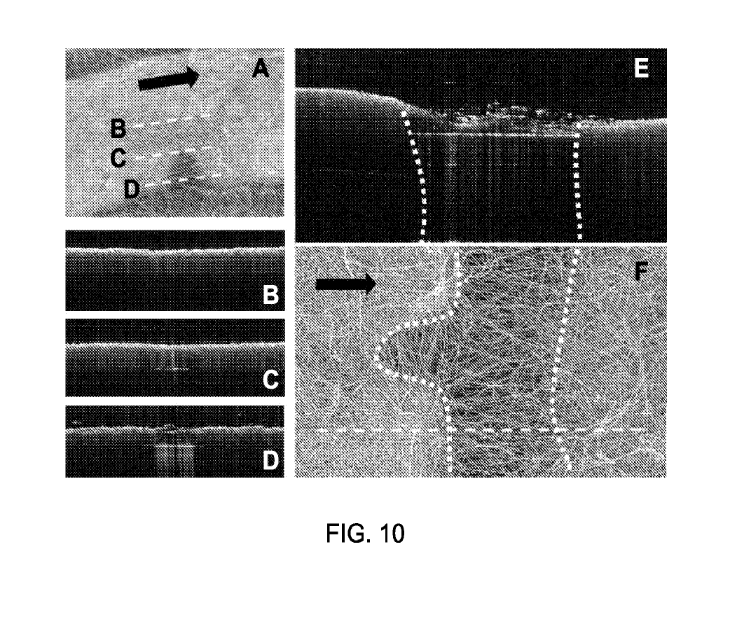

The complexity of the electrospun scaffold was studied in detail by OCT, SEM and phase contrast imaging. The different parts of the construct were imaged and a correlation between OCT and SEM images was established (FIG. 10).

Phase Contrast Microscopy was carried out using an inverted Olympus CK40 microscope and SEM was performed using a Philips X-L 20 microscope. The OCT system used for this study was equipped with a laser source (Santec HSL-2000) operated at 10 kHz rate with 10 mW output power and a central wavelength of 1300 nm.

### 3.5 Cell Viability and Cell Morphology on PLGA Microfabricated Membranes and Niches

Cell viability on the electrospun rings was evaluated using the MTT (3-(dimethylthiazol-2-yl)-2,5-diphenyltetrazolium bromide) assay. Rabbit limbal fibroblasts were seeded on PLGA rings, PLGA plain membranes and glass coverslips as positive controls. RLF were seeded specifically in the areas of the micropockets under a dissection microscope (Wild Heerbrugg M 3Z) and using an Eppendorf Micropipette (0.5-10 μl range) dispensing volumes of 3-6 μl in each niche. For plain scaffolds and glass coverslip controls cells were seeded in the same way. The cells were kept for 6 days in an incubator at 37° C. and 5% CO₂. After that, the media was removed and RLF were washed with PBS. MTT (0.5 mg/ml) was added for 40 minutes (37° C.). Acidified isopropanol (1 μl HCl in 1 ml isopropanol) was then added for dissolving the formazan crystals resulting from MTT reduction. The samples were measured using a BIO-TEK ELx 800 microplate reader at the wavelength of 540 nm and (referenced at 630 nm).

For fluorescence imaging rabbit limbal fibroblasts were seeded on the scaffolds at a concentration of 1×10⁵cells/ring and epithelial cells were seeded at a concentration of 5×10⁴ cells/ring. RLF were stained with phalloidin-TRITC or phalloidin-FITC (to label actin filaments) and epithelial cells well also labeled with anti-vinculin staining. Cells were fixed in 10% formalin in PBS for 30 min at room temperature. Phalloidin-FITC was then added 1:500 in PBS for 30 min. RLE were stained using monoclonal anti-vinculin produced in mouse (Sigma Aldrich). After fixation with 3.7% formaldehyde the samples were permeabilized with Triton X-100 (0.1%) for 30 minutes and then washed with PBS. The cells were blocked with 10% goat serum (Sigma Aldrich) for 1 hour and then incubated with primary antibody diluted (1:150 in 1% goat serum) for another hour. The scaffolds were then washed with PBS and incubated with biotinylated secondary anti-mouse antibody (1:1000 in 1% goat serum, Vector Labs) for 1hour at room temperature and further washed with PBS. Finally the epithelial cells were incubated with tertiary antibody FITC-streptavidin (1:100 in 1% goat serum, Vector Labs) for 30 min at room temperature and then treated with the nuclear staining DAPI and Phalloidin-TRITC.

Cells were imaged inside and outside the microfeatures using a confocal scanning microscope (Carl Zeiss LSM510-META, Germany) and an ImageXpress (Axon Instrumen, USA).

### 3.6. Rabbit Limbal Explant Outgrowth on PLGA Membranes

Previous the inclusion of the rabbit limbal explants in the ex vivo model a study of the cell outgrowth from the explants was performed by placing explants on fibrin-glue treated micropockets. The microfabricated scaffolds were treated with 10 μl fibrin+10 μl thrombin and the explants were placed using a needle using a dissection microscope. Explant outgrowth was analysed using confocal microscopy and SEM.

For SEM imaging cells were washed with PBS and them fixed in 2.5% glutaraldehyde for 1 hour followed by dehydration with increasing concentration gradients of ethanol (from 35% to 100%). The samples were dried using hexamethyldisilazane (HMDS): ethanol 1:1 for 1 h and finally treated with 100% HMDS for 5 min. The electrospun rings were sputter-coated with gold (emscope SC 500 coater) and analysed by a Philips X-L 20 microscope. Explant outgrowth was also studied using phalloidin-TRITC/DAPI and BrdU (Bromodeoxyuridine) staining.

### 3.7. Immunostaining

PLGA electrospun rings seeded with limbal epithelial cells were fixed and inmunolabeled after 24 hours and after 2 weeks in culture. Limbal explants were fixed after 2 and 3 weeks in culture. In both cases the samples were fixed with formalin 3.7%, permeabilized with Triton-X 0.5% for 20 min and blocked with 10% goat serum during 1 hour. Samples were incubated with mouse monoclonal antibody cytokeratin 3 (CK3, Merck Millipore) and P63 (Merck Millipore) in 1% goat's serum overnight at 4° C. After PBS washes, the rings were treated with biotinylated secondary anti-mouse antibody (1:1000 in 1% goat serum, Vector Labs) for 1 hour at room temperature and tertiary antibody FITC-streptavidin (1:100 in 1% goat serum, Vector Labs) for 30 min at room temperature; samples were finally treated treated with the nuclear staining DAPI.

Immunohistochemistry procedures were performed in the histology sections obtained from the organ culture models. The sections were dewaxed in xylene and rehydrated in 100% ethanol, 70% ethanol and distilled water. The sections were then delineated with a Dako pen and treated with 0.05% trypsin (Aldrich) for 20 minutes (37° C.). After washing with PBS the samples were blocked with 10% goat's serum for 1 hour and treated with CK3 and P63 as described in the paragraph above.

### 4. Results

### 4.1 Electrospun Ring Complexity and Fibre Alignment

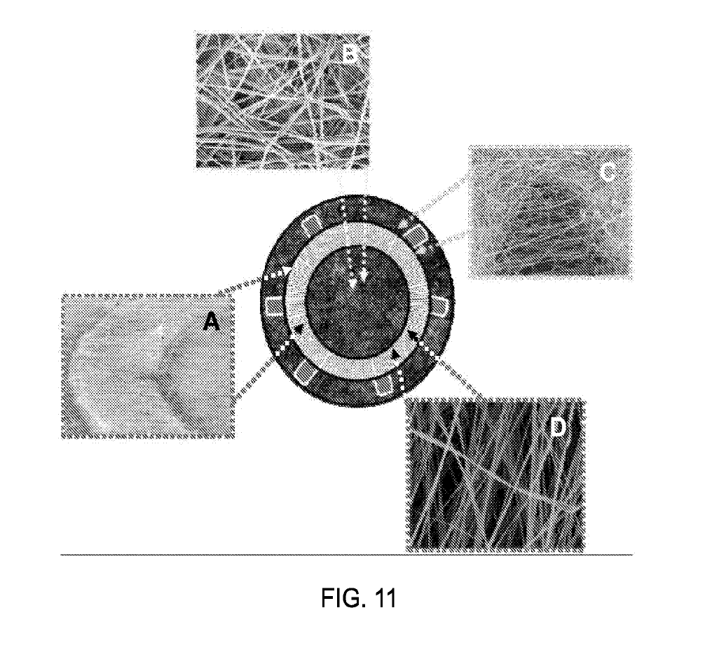

The electrospinning stereolithography-enabled process used to create the 3D corneal rings allows the control and creation of intricate structures with distinguished parts showing different fibre density and alignment. The underlaying structure provided by the scaffold fibres plays an important role in cell morphology hence the importance of describing the structure of the ring scaffolds in detail. The rings are formed by 4 important parts (see FIG. 11). Part A is the outer ring which is a high density mash of fibres randomly orientated; part B is the centre of the hybrid membrane which again shows high density of fibres and random alignment; part C corresponds to the microfabricated pockets which in this case are horse-shoe shaped. Finally, part D corresponds to the area connecting the outer ring and the central membrane; the length of this area can be controlled by controlling the high of the PEGDA collectors as reported above. Part D is formed by aligned parallel fibres. The fibres in part D are perpendicular to the fibres in part C (niche).

The differences in fibre density in the four areas of the ring-scaffolds were studied using OCT. The results were correlated with SEM imaging as showed in FIG. 10. The samples were scanned in different directions. First of all, the areas of the niches were chosen (image 10A) and different scans were performed in parallel directions towards the centre of the construct (directions highlighted with the yellow lines (B, C, D) in FIG. 10A). The consecutive scans B, C and D showed differences in density in areas previous the appearance of the the niche (B), areas in the beginning of the niche (C) and areas at the end of the niche (D). A second kind of scan perpendicular to the niche was performed (direction of scan showed with a red arrow in image 2F). In the second scan we were able to follow the differences in density of the ring, the area connecting ring and membrane and the central area of the membrane. The difference of densities is clear when correlating OCT scans (10E) with SEM images (10F).

### 4.2 Cell Viability and Morphology on PLGA Plain Scaffolds and Electrospun Rings

The experimental data described above discloses viability studies performed in both ring and plain PLGA scaffolds demonstrating that cells were viable in both type of membranes and they proliferate at comparable rate. In the present experiment cells were seeded specifically in the areas of the pockets and in a similar manner they were distributed in the plain membranes. MTT assay didn't show significative differences between both types of membranes. A closer analysis of the MTT-treated membranes showed differences regarding the distribution of cells in the membranes after 6 days in culture. Cells located in the pockets seemed to be guided towards the centre of the scaffolds following the direction marked by the underlaying electrospun fibres in the part connecting the niche and the central scaffold (FIG. 10B). This tendency wasn't observed for the cells placed on the plain scaffolds (FIG. 10D).

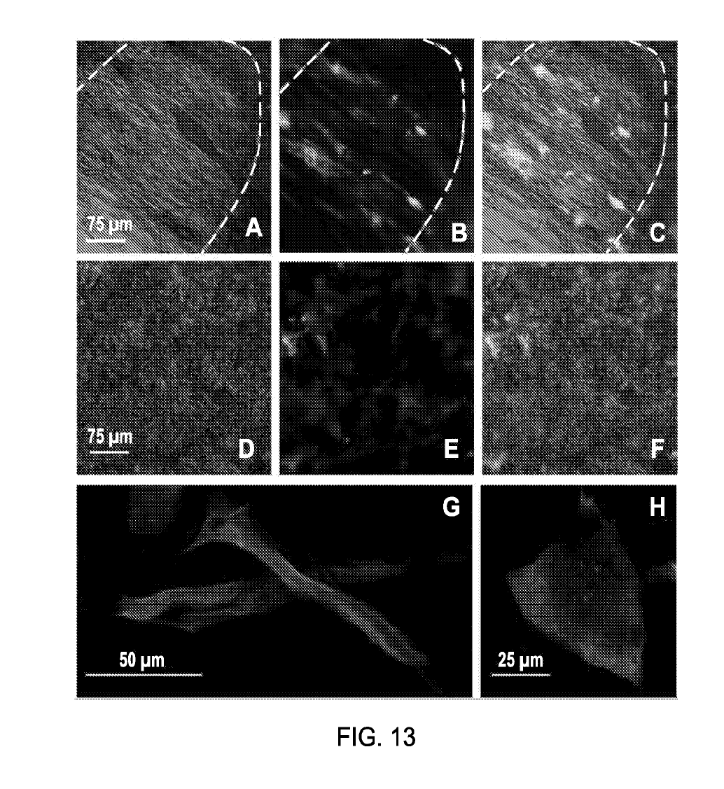

Cell morphology was also studied inside and outside the micropockets. Both rabbit limbal fibroblasts and epithelial cells presented different morphology in the different areas. The morphology was dictated by the underlying structure. Images A-C in FIG. 13 show RLF stained with phalloidin-FITC extended across the niche structure and following the parallel orientated fibres. Images D-F show RLF stained with phalloidin-FITC in a random-fibre area of the scaffold. The fibroblast inside the micropockets show a more elongated morphology. The same effect was observed for epithelial cells. Image G in FIG. 4 corresponds to a confocal image of epithelial cells taken inside of the micropocket and Image H corresponds to an epithelial cells in the central area of the membrane. In both cases the cells were stained for vinculin (green, showing phocal adhesion points) and for phalloidin-TRITC (red).

### 4.3. Characterisation of Rabbit Limbal Epithelial Cells on Electrospun Ring Scaffolds

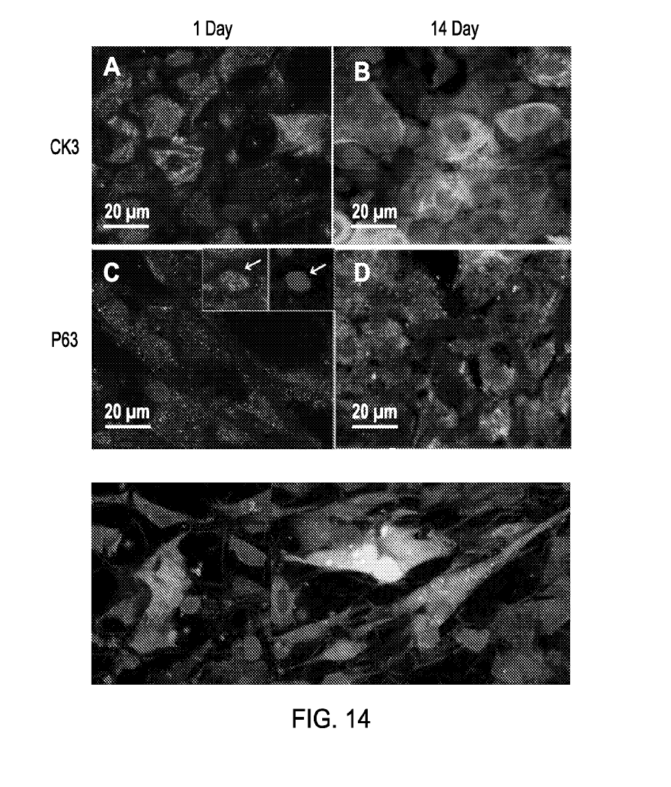

Rabbit limbal epithelial cells were seeded on PLGA rings at passage 1 and immunolabelled with CK3 and P63 at different time-points. CK3 is a cytokeration expressed in corneal epithelium together with CK12 and P63 is a stem cell and transient amplifying cell marker. No differences in the expression of both CK3 and P63 were observed inside and outside the micropockets. P63 was positive in the nuclei but also in the cytoplasm (FIG. 14C, 14D) and Ck3 was observed in the cytoplasm (FIGS. 14A, 14B). The markers were expressed at the two different studied time points. After 14 days the cells increased in number forming a 80-90% confluent monolayer and demonstrating the ability of limbal epithelial cells to proliferate in our constructs.

### 4.4. Cell Outgrowth from Rabbit Limbal Explants Located on Electrospun Pockets

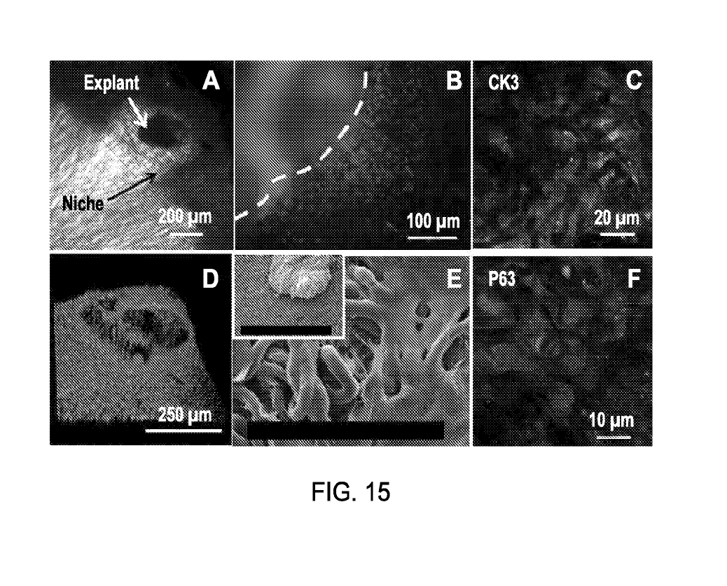

Rabbit limbal explants were located in PLGA rings previously treated with fibrin glue; the pieces of tissue were placed directly on the electrospun niches (FIG. 15A). Cell outgrowth was studied after 2 and 3 weeks of culture. The morphology of the cells coming out from the explants was assessed by fluorescence microscopy and SEM (images 5B and 5E). Cells were positive for CK3 and P63 staining (FIG. 15C and 15F). Figure D shows a confocal z-stack of an explant placed on an electrospun niche; the cells coming out from the explant were BrdU positive which demonstrated the presence of proliferative cells.

### 4.5. Cell Outgrowth and Transfer from PLGA Electrospun Rings in 3D Organ Culture Models

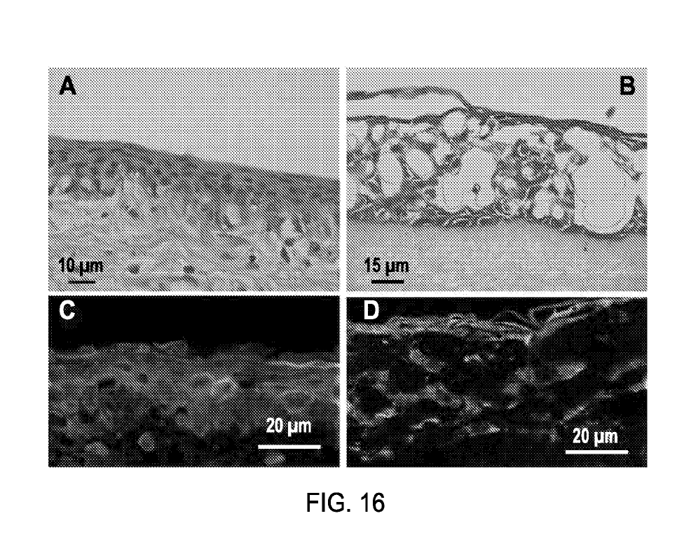

PLGA rings seeded with limbal epithelial cells were kept in organ cultured for 4 weeks as previously described. The samples were section and analysed by histology and immunohistochemistry. Epithelial cell transfer from PLGA rings to the deliberately denuded corneas was achieved both placing cells facing down and facing up either submerging the whole cornea or keeping the organ culture at air-liquid interface. FIG. 16 compares a fresh rabbit corneal epithelium (A) with the cell transfer achieved by a PLGA scaffold with epithelial cells facing up on a denuded cornea kept at air-liquid interface (B). Cells seeded in the pockets were able to migrate towards the centre of the cornea starting to form a new epithelium. Immunochemistry results (FIG. 16C, D) demonstrated that the cells transferred were corneal epithelial cells since they were positive for CK3 staining (green).

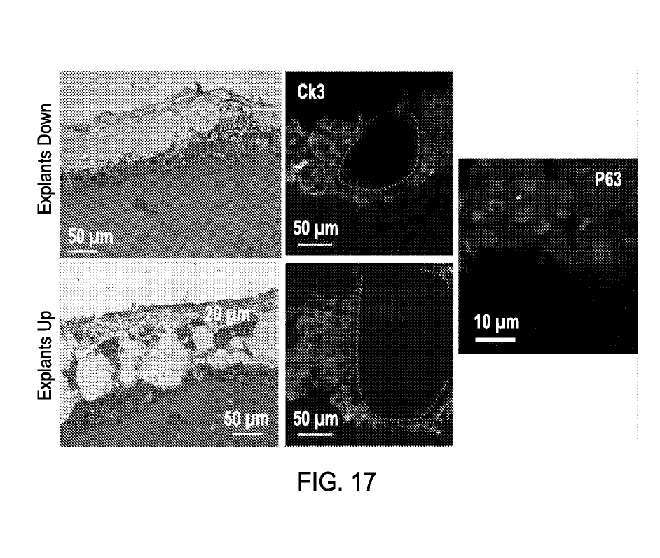

Transfer experiments were also performed using limbal explants. Electrospun rings with explants were placed on wounded corneas both facing up/down and using fibrin glue. Cell outgrowth was observed for both conditions as exemplified in FIG. 17. H&E staining showed the formation of a new epithelium along all the cornea; in some cases the regenerated epithelium was very similar to the multilayer epithelium presented by an intact rabbit cornea. Cell outgrowth proved to be better when coating the scaffold with a thin layer of fibrin glue. Histological sections were immunolabelled with CK3 demonstrating that the cells coming out from the explants were corneal epithelial cells. Moreover, P63 staining was positive for all the cases.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### BIBLIOGRAPHY

Blackwood, K. A., R. McKean, et al. (2008). “Development of biodegradable electrospun scaffolds for dermal replacement.” Biomaterials 29(21): 3091-3104.
Bray, D. F., J. Bagu, et al. (1993). “Comparison of hexamethyldisilazane (HMDS), Peldri II, and critical-point drying methods for scanning electron microscopy of biological specimens.” Microscopy Research and Technique 26(6): 489-495.
Cotsarelis, G., S.-Z. Cheng, et al. (1989). “Existence of slow-cycling limbal epithelial basal cells that can be preferentially stimulated to proliferate: Implications on epithelial stem cells.” Cell 57(2): 201-209.
Deshpande, P., R. McKean, et al. “Using poly(lactide-co-glycolide) electrospun scaffolds to deliver cultured epithelial cells to the cornea.” Regenerative Medicine 5(3): 395-401.
Dravida, S., S. Gaddipati, et al. (2008). “A biomimetic scaffold for culturing limbal stem cells: a promising alternative for clinical transplantation.” Journal of Tissue Engineering and Regenerative Medicine 2(5): 263-271.
Ebato, B., J. Friend, et al. (1987). “Comparison of central and peripheral human corneal epithelium in tissue culture.” Investigative Ophthalmology & Visual Science 28(9): 1450-6.
Fuchs, E., T. Tumbar, et al. (2004). “Socializing with the Neighbors: Stem Cells and Their Niche.” Cell 116(6): 769-778.
Li, D., G. Ouyang, et al. (2005). “Collecting Electrospun Nanofibers with Patterned Electrodes.” Nano Letters 5(5): 913-916.
Liang, D., B. S. Hsiao, et al. (2007). “Functional electrospun nanofibrous scaffolds for biomedical applications.” Advanced Drug Delivery Reviews 59(14): 1392-1412.
Matthews, J. A., G. E. Wnek, et al. (2002). “Electrospinning of Collagen Nanofibers.” Biomacromolecules 3(2): 232-238.
McLaughlin, C. R., M. C. Acosta, et al. “Regeneration of functional nerves within full thickness collagenâ [symbol] “phosphorylcholine corneal substitute implants in guinea pigs.” Biomaterials 31 (10): 2770-2778.
Shin, Y. M., M. M. Hohman, et al. (2001). “Electrospinning: A whipping fluid jet generates submicron polymer fibers.” Applied Physics Letters 78(8): 1149-1151.
Sitalakshmi, G., B. Sudha, et al. (2008). “Ex Vivo Cultivation of Corneal Limbal Epithelial Cells in a Thermoreversible Polymer (Mebiol Gel) and Their Transplantation in Rabbits: An Animal Model.” Tissue Engineering Part A 15(2): 407-415.
Vaquette, C. and J. J. Cooper-White “Increasing electrospun scaffold pore size with tailored collectors for improved cell penetration.” Acta Biomaterialia 7(6): 2544-2557.
Zhang, D. and J. Chang (2007). “Patterning of Electrospun Fibers Using Electroconductive Templates.” Advanced Materials 19(21): 3664-3667.
Zhang, D. and J. Chang (2008). “Electrospinning of Three-Dimensional Nanofibrous Tubes with Controllable Architectures.” Nano Letters 8(10): 3283-3287.
Zong, X., H. Bien, et al. (2005). “Electrospun fine-textured scaffolds for heart tissue constructs.” Biomaterials 26(26): 5330-5338.
Zussman, E., A. Theron, et al. (2003). Formation of nanofiber crossbars in electrospinning, AIP.

1. A method for producing an electrospun scaffold, comprising electrospinning a polymer or co-polymer onto a template comprising a conductive collector having a three dimensional pattern thereon, wherein said electrospun polymer or co-polymer preferentially deposits onto said three dimensional pattern.

2. The method according to claim 1, wherein said three dimensional pattern is non-conductive.

3. The method according to claim 1 or claim 2, wherein said three dimensional pattern is dimensioned to provide an electrospun scaffold having at least one cavity therein capable of acting as a stem cell niche.

4. The method according to any one of the preceding claims wherein said polymer or co-polymer is biodegradable.

5. The method according to claim 4, wherein said biodegradable polymer or copolymer is biocompatible.

6. The method according to claim 4 or claim 5, wherein said biodegradable polymer is a collagen, a poly alpha ester, a polyorthoester or a polyanhydride or a copolymer thereof.

7. The method according to claim 6, wherein said biodegradable polymer or copolymer is cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde or a copolymer thereof.

8. The method according to claim 6, wherein said polymer is polylactate acid.

9. The method according to claim 6, wherein said polymer is polyglycolic acid.

10. The method according to claim 6, wherein said copolymer is a copolymer of polylactate acid and polyglycolic acid.

11. The method according to claim 10, wherein said copolymer is a poly(D,L-lactic-co-glycolide).

12. The method according to claim 11, wherein said poly(D,L-lactic-co-glycolide) has a ratio of 75:25 lactide to glycolide.

13. The method according to claim 11, wherein said poly(D,L-lactic-co-glycolide) has a ratio of 50:50 lactide to glycolide.

14. The method according to any one of the preceding claims, wherein said conductive collector is an aluminium sheet.

15. The method according to any one of the preceding claims, wherein said three dimensional pattern is formed on said carrier by microfabrication.

16. The method according to claim 15, wherein said microfabrication is microstereolithography.

17. The method according to any one of the preceding claims, wherein said three dimensional pattern is formed from polyethelyne glycol.

18. An electrospun scaffold having at least one cavity therein capable of acting as a stem cell niche produced in accordance with the method of any one of claims 1 to 17.

19. An electrospun scaffold, comprising a biodegradable polymer or co-polymer, wherein said scaffold comprises at least one cavity therein capable of acting as a stem cell niche.

20. The electrospun scaffold according to claim 19, wherein said biodegradable polymer or copolymer is biocompatible.

21. The electrospun scaffold according to claim 19 or claim 20, wherein said biodegradable polymer is a collagen, a poly alpha ester, a polyorthoester or a polyanhydride or a copolymer thereof.

22. The electrospun scaffold according to claim 21, wherein said biodegradable polymer or copolymer is cellulose ether, cellulose, cellulosic ester, fluorinated polyethylene, phenolic, oly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde or a copolymer thereof.

23. The electrospun scaffold according to claim 21, wherein said polymer is polylactate acid.

24. The electrospun scaffold according to claim 21, wherein said polymer is polyglycolic acid.

25. The electrospun scaffold according to claim 21, wherein said copolymer is a copolymer of polylactate acid and polyglycolic acid.

26. The electrospun scaffold according to claim 25, wherein said copolymer is a poly(D, L-lactic-co-glycolide).

27. The electrospun scaffold according to claim 26, wherein said poly(D,L-lactic-co-glycolide) has a ratio of 75:25 lactide to glycolide.

28. The electrospun scaffold according to claim 26, wherein said poly(D,L-lactic-co-glycolide) has a ratio of 50:50 lactide to glycolide.

29. The electrospun scaffold according to any one of claims 18 to 28, wherein said scaffold further comprises stem cells.

30. The electrospun scaffold according to claim 29 wherein said stem cells are limbal stem cells.

31. The electrospun scaffold according to claim 29 wherein said stem cells are mesenchymal stem cells.

32. An electrospun scaffold according to any one of claims 18 to 31, for use as a medicament.

33. An electrospun scaffold according to any one of claims 18 to 31, for use in corneal replacement.

34. An electrospun scaffold according to any one of claims 18 to 31, for use in the treatment of ocular injury.

35. An electrospun scaffold according to any one of claims 18 to 31, for use in the treatment of a wound.

36. An electrospun scaffold according to claim 35, wherein said wound is a chronic wound or wherein said wound is an acute wound.

37. An electrospun scaffold as described herein with reference to the accompanying drawings.

38. A method for producing an electrospun scaffold as described herein with reference to the accompanying drawings.
